(19) United States
(12) Patent Application Publication
(10) Pub. No.: US 2026/0249019 A1
(43) Pub. Date: Aug. 27, 2026

(54) A cap assembly of a medicament delivery device

(71) Applicant: SHL Medical AG, Zug (CH)

(72) Inventors: René Zander, Zug (CH); Oscar Alexandersson, Nacka Strand (SE); Christian Forrer, Wil (CH); Tobias Rütsche, Wil (CH)

(21) Appl. No.: 19/489,632

(22) PCT Filed: Jun. 13, 2024
(86) PCT No.: PCT/EP2024/066444

§ 371 Date: Dec. 3, 2025

Related U.S. Application Data
(60) Provisional application No. 63/523,735, filed on Jun. 28, 2023.
(60) Provisional application No. 63/524,290, filed on Jun. 30, 2023.

(30) Foreign Application Priority Data
Nov. 24, 2023 (EP) 23211978.4

(51) Int. Cl. A61M 5/32; A61M 5/20

(52) U.S. Cl. CPC A61M 5/3204; A61M 5/2033; A61M 5/3216

(57) ABSTRACT

The present disclosure provides a cap assembly of a medicament delivery device. The cap assembly includes a cap body extending along a longitudinal axis between a proximal end and a distal end. The cap assembly further includes a delivery member shield remover extending along the longitudinal axis between a proximal end and a distal end. The delivery member shield remover is positioned within the cap body, and the delivery member shield remover comprises a grabber configured to be engaged with a delivery member shield of the medicament delivery device such that a user of the medicament delivery device can remove the delivery member shield when remove the cap assembly from a housing of the medicament delivery device. The cap body comprises a proximally directed surface adjacent to a distally directed surface of the delivery member shield remover The cap body comprises a user-accessible surface configured to be gripped by the user of the medicament delivery device such that the user can remove the cap assembly from the housing of the medicament delivery device via gripping on the user-accessible surface. The distally directed surface of the delivery member shield remover is positioned closer to the distal end of the delivery member shield remover than the proximal end of the delivery member shield remover.

### TECHNICAL FIELD

The present disclosure generally relates to a cap assembly of a medicament delivery device, and particularly to a cap assembly of a medicament delivery device that comprises a delivery member shield remover.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency situation, the users can easily and properly use the medicament delivery devices.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a cap assembly of a medicament delivery device. The cap assembly comprising: a cap body extending along a longitudinal axis between a proximal end and a distal end; and a delivery member shield remover extending along the longitudinal axis between a proximal end and a distal end. The delivery member shield remover is positioned within the cap body. The delivery member shield remover comprises a grabber configured to be engaged with a delivery member shield of the medicament delivery device such that a user of the medicament delivery device can remove the delivery member shield when remove the cap assembly from a housing of the medicament delivery device. The cap body comprises a proximally directed surface adjacent to a distally directed surface of the delivery member shield remover. The cap body comprises a user-accessible surface configured to be gripped by the user of the medicament delivery device such that the user can remove the cap assembly from the housing of the medicament delivery device via gripping on the user-accessible surface. The distally directed surface of the delivery member shield remover is positioned closer to the distal end of the delivery member shield remover than the proximal end of the delivery member shield remover.

Preferably, according to one embodiment, the proximally directed surface and the user-accessible surface are immovable relative to one another. For example, the proximally directed surface and the user-accessible surface are two parts of one single component such that the proximally directed surface and the user-accessible surface are neither axially movable nor rotatable relative to one another. Thus, the force from the user can be transferred to the delivery member shield remover efficiently.

Preferably, according to one embodiment, the distal end of the delivery member shield remover comprises the distally directed surface of the delivery member shield remover.

Preferably, according to one embodiment, the grabber of the delivery member shield remover comprises a proximally directed surface configured to be engaged with the delivery member shield of the medicament delivery device.

Preferably, according to one embodiment, the delivery member shield remover comprises at least two grabbers lined up to one another in the direction of the longitudinal axis.

Preferably, according to one embodiment, the cap body comprises a distally directed surface adjacent to the proximal end of the delivery member shield remover.

Preferably, according to one embodiment, the cap body comprises a flexible arm extending towards the proximal end of the cap body. The flexible arm comprises the proximally directed surface of the cap body such that the delivery member shield remover is attached to the cap body via a snap fit.

Preferably, according to one embodiment, the cap body comprises an inner body and an outer body being coaxial to the inner body. The outer body comprises the user-accessible surface. The inner body comprises the distally directed surface.

Preferably, according to one embodiment, the outer body comprises a proximal wall extending in a direction transverse to the longitudinal axis. The inner body comprises a body wall extending from the proximal wall of the outer body along the longitudinal axis towards the distal end of the cap body. The body wall of the inner body defines a cannel. The delivery member shield remover is snuggly fit into the cannel of the inner body.

Preferably, according to one embodiment, the inner body comprises a rib extending from the body wall towards the longitudinal axis. The rib comprises the distally directed surface of the cap body.

Preferably, according to one embodiment, the inner body comprises the flexible arm protruding from the body wall.

Preferably, according to one embodiment, a cut-out/recess is arranged in the body wall of the inner body. The flexible arm extends from an edge of the cut-out/recess towards the proximal end of the cap body.

Preferably, according to one embodiment, the delivery member shield remover is positioned between the rib of the inner body and the flexible arm of the inner body.

Preferably, according to one embodiment, the outer body comprises a fixture configured to be releasably attached to the housing of the medicament delivery device.

Preferably, according to one embodiment, the grabber is positioned closer to the distal end of the delivery member shield remover than the proximal end of the delivery shield remover.

Preferably, according to one embodiment, the delivery member shield remover comprises an inclined surface inclined towards the longitudinal axis from the distally directed surface of the delivery member shield remover.

Another aspect of the invention provides a medicament delivery device comprising the cap assembly as mentioned above.

Preferably, according to one embodiment, the medicament delivery device comprises a medicament container.

Preferably, according to one embodiment, the medicament container is made of glass or plastic.

Preferably, according to one embodiment, the medicament container is a syringe, a cartridge or a collapsible bag.

Preferably, according to one embodiment, the medicament container is integral with a medicament delivery member.

Preferably, according to one embodiment, the medicament container is configured to be connected to a medicament delivery member before a medicament delivery operation taking place.

Preferably, according to one embodiment, the medicament container comprises multiple chambers to container different substances respectively.

Preferably, according to one embodiment, the medicament delivery device is an autoinjector, a manual injector, a safety syringe, an inhaler, a sprayer, or an infusion pump.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIGS. 1A and 1B are perspective views of a medicament delivery device;

FIG. 2 is a perspective view of an example of an administration mechanism of the medicament delivery device in FIGS. 1A and 1B;

FIG. 3 shows the administration mechanism in FIG. 2 with the medicament delivery member cover removed exposing a medicament container holder;

FIGS. 4 and 5 show perspective views of certain components of the administration mechanism in FIG. 2;

FIGS. 6 and 7 show additional components of the administration mechanism in FIG. 2;

FIG. 8 is a longitudinal section of a plunger holder and plunger rod of the administration mechanism, the section being at right angle to the longitudinal section in FIG. 8; and

FIGS. 9A-B show longitudinal sections of the medicament delivery device in FIG. 1A-B in different stages of operation.

FIGS. 10-15 show perspective views of certain components of the medicament delivery device in FIG. 2;

FIGS. 16-19 show perspective views of the cap assembly of the invention; and

FIG. 20 shows a cross-section view of the cap assembly of the invention.

FIGS. 21A-D show cross-section views of a delivery member shield remover of FIG. 20 with different distally directed surface respectively.

FIG. 22 shows a perspective view of a delivery member shield remover of FIG. 20 in another embodiment.

### DETAILED DESCRIPTION

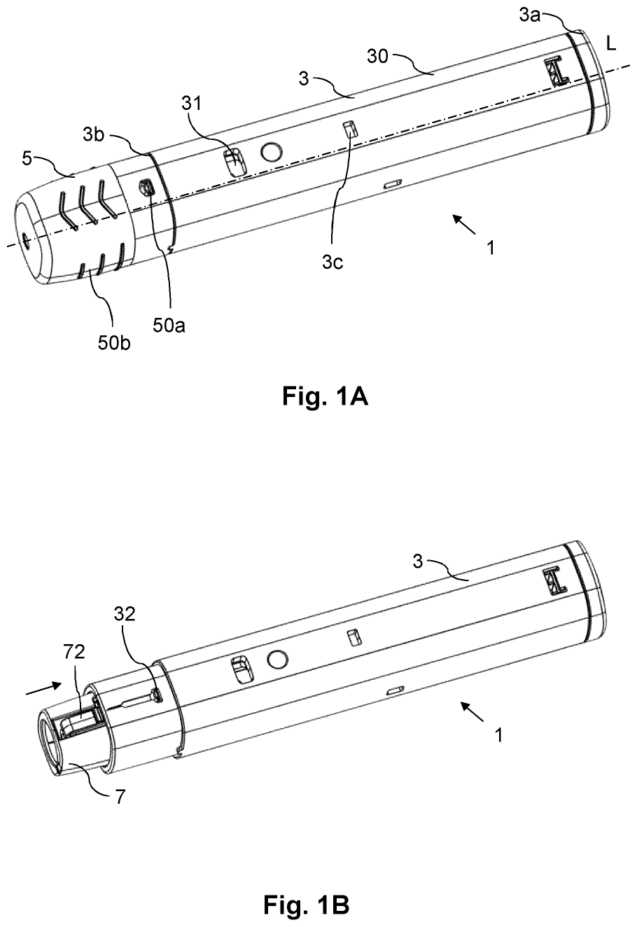

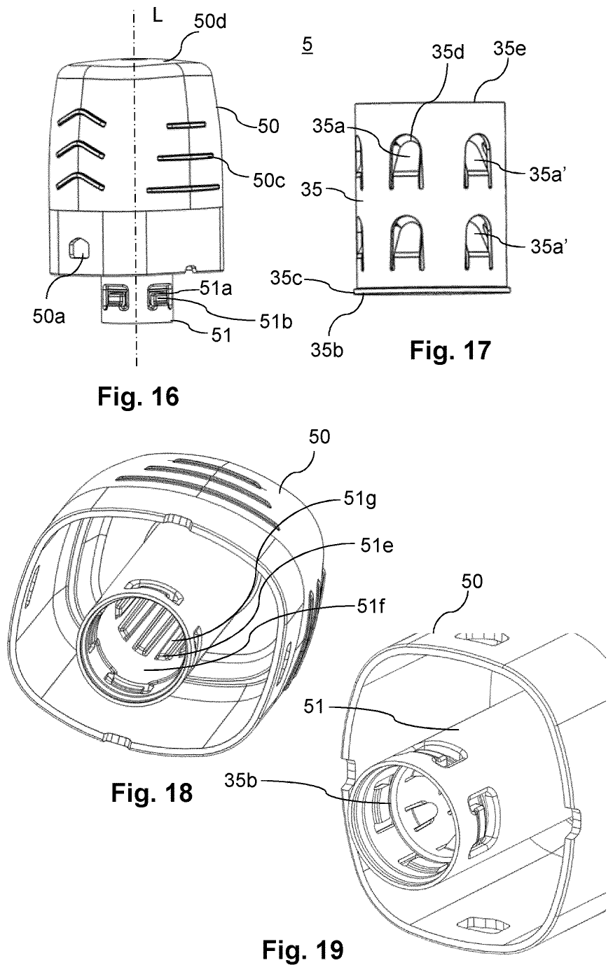

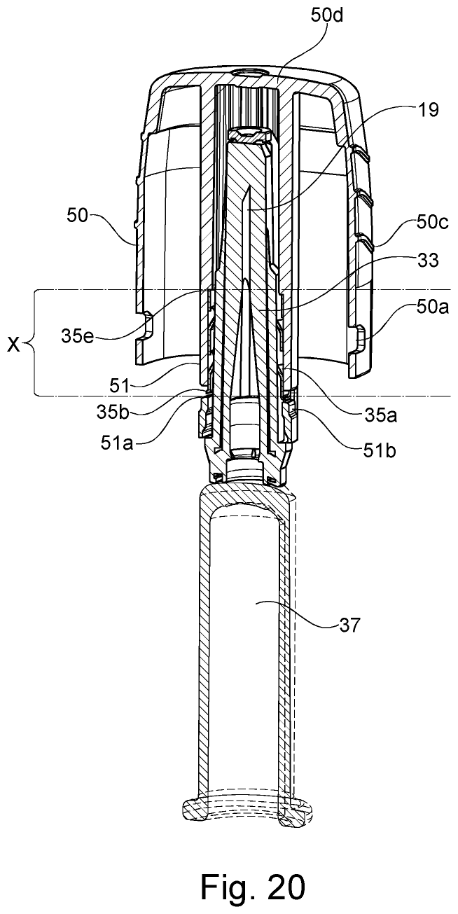

FIGS. 1-20 illustrate a cap assembly 5 of a medicament delivery device of the invention. The cap assembly 5 comprises a cap body 50, 51 and a delivery member shield remover 35, as shown in FIGS. 16-17. The cap body 50, 51 extends along a longitudinal axis L between a proximal end and a distal end. The delivery member shield remover 35 extends along the longitudinal axis L between a proximal end and a distal end. The delivery member shield remover 35 is positioned within the cap body 50, 51, as shown in FIGS. 19-20. The delivery member shield remover 35 comprises a grabber 35a configured to be engaged with a delivery member shield 33 of the medicament delivery device such that a user of the medicament delivery device can remove the delivery member shield 33 when remove the cap assembly 5 from a housing 3 of the medicament delivery device. The delivery member shield can be made of a flexible material and/or a rigid material and/or be a combination of a rigid shield and a flexible shield. The grabber 35a can be designed to stab into the flexible shield and/or engaged with an edge/a protrusion/a recess/cut-out of the delivery member shield. The delivery member shield remover 35 can be made of metal or plastic. In one example where the delivery member shield remover 35 is made of metal, the delivery member shield remover 35 can be made of a sheet metal or a metal tube.

The cap body 50, 51 comprises a proximally directed surface 51a adjacent to a distally directed surface 35b; 35b′; 35b″; 35b″′ of the delivery member shield remover 35. The cap body 50, 51 comprises a user-accessible surface 50c configured to be gripped by the user of the medicament delivery device such that the user can remove the cap assembly 5 from the housing 3 of the medicament delivery device via gripping on the user-accessible surface 50c. The distally directed surface 35b; 35b′; 35b″; 35b″′ of the delivery member shield remover 35 is positioned closer to the distal end of the delivery member shield remover 35 than the proximal end of the delivery member shield remover 35.

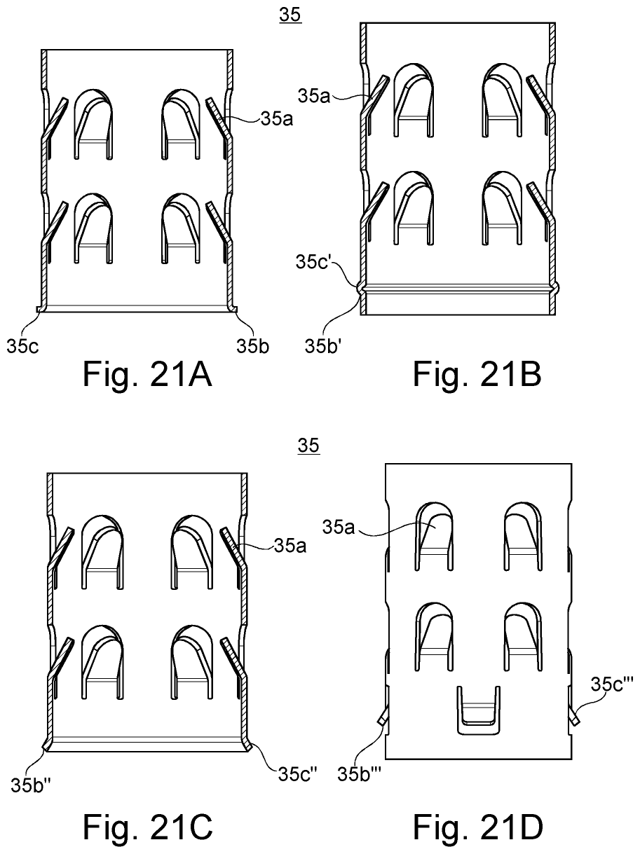

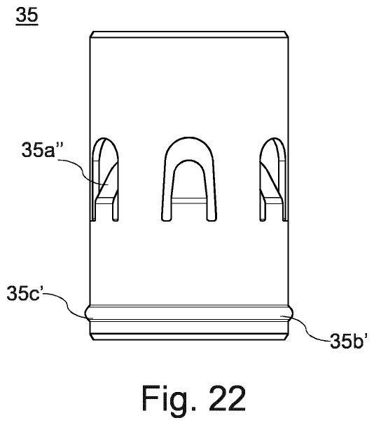

In a preferred example, the distal end of the delivery member shield remover 35 comprises the distally directed surface 35b; 35b″of the delivery member shield remover 35, as shown in FIG. 21A and FIG. 21C. Alternatively, or additionally, the delivery member shield remover 35 comprises a ledge 35c; 35c′; 35c″′ extending radially outward that comprises the distally directed surface 35b; 35b′. In one example, as shown in FIG. 21B and FIG. 22, the ledge 35c′ is a rim 35c′ circumferentially extends around the delivery member shield remover 35. Furthermore, in another example, as shown in FIG. 21A, the ledge 35c is arranged at the distal end of the delivery member shield remover 35. Alternatively, the ledge 35c′ is positioned between the grabber 35a and the distal end of the delivery member shield remover 35, as shown in FIG. 21B, and FIG. 22. Alternatively, the delivery member shield remover 35 comprises a snap arm 35c″′ extending radially outwards away from the longitudinal axis L; in this example, the snap arm 35c″′ comprises the distally directed surface 35b″′, as shown in FIG. 21D. In one example, the ledge 35c is generally perpendicular to the longitudinal axis L. Alternatively, the ledge 35c″′ comprises an inclined surface 35c″ surface, as shown in FIG. 21C.

In another example, the grabber 35a of the delivery member shield remover 35 comprises a proximally directed surface 35d configured to be engaged with the delivery member shield 33 of the medicament delivery device. Preferably, the delivery member shield remover 35 comprises at least two grabbers 35a′, 35a′ lined up to one another in the direction of the longitudinal axis L. As a result, the removal force can be evenly distributed on the delivery member shield 33. Alternatively, the grabbers 35a″ are arranged circumferentially around the delivery member shield remover 35 and formed a single row as shown in FIG. 22. In this example, the cost of the cap assembly can be reduced.

In another example, the cap body 50, 51 comprises a distally directed surface 51e adjacent to the proximal end 35e of the delivery member shield remover 35e.

In a preferred example, the cap body 50, 51 comprises a flexible arm 51b extending towards the proximal end of the cap body 50, 51. The flexible arm 51b comprises the proximally directed surface 51a of the cap body 50, 51 such that the delivery member shield remover 35 is attached to the cap body 50, 51 via a snap fit.

In another example, the cap body 50, 51 comprises an inner body 51 and an outer body 50 being coaxial to the inner body 51. The outer body 50 comprises the user-accessible surface 50c. The inner body 51 comprises the distally directed surface 51a. Preferably, the outer body 50 comprises a proximal wall 50d extending in a direction transverse to the longitudinal axis L. The inner body 51 comprises a body wall extending from the proximal wall 51d of the outer body 50 along the longitudinal axis L towards the distal end of the cap body 50, 51. The body wall of the inner body 51 defines a cannel 51f. The delivery member shield remover 35 is snuggly fit into the cannel 51f of the inner body 51, as shown in FIG. 19.

In another example, the inner body 51 comprises a rib 51g extending from the body wall towards the longitudinal axis L. The rib 51g comprises the distally directed surface 51e of the cap body 50, 51. In another example, the inner body 51 comprises the flexible arm 51b protruding from the body wall. Preferably, a cut-out/recess is arranged in the body wall of the inner body 51. The flexible arm 51b extends from an edge of the cut-out/recess towards the proximal end of the cap body 50, 51. In another example, the delivery member shield remover 35 is positioned between the rib 51g of the inner body 51 and the flexible arm 51b of the inner body 51, as shown in FIG. 20 with a highlighted position X.

In another example, the outer body 50 comprises a fixture 50a configured to be releasably attached to the housing 3 of the medicament delivery device.

In another example, the grabber 35a is positioned closer to the distal end of the delivery member shield remover 35 than the proximal end of the delivery shield remover 35. As the delivery member shield remover 35 is connected to the cap body 50, 51 via the distally directed surface 35b; 35b′; 35b″; 35b″′ of the delivery member shield remover 35 to enable the delivery member shield remover 35 to be removed together with the cap body 50, 51. By arranging the grabber 35a of the delivery member shield remover 35 closer to the distal end of the delivery member shield remover 35, the size of the delivery member shield remover 35 can be small, thus, the material cost of the delivery member shield remover 35 can be reduced.

In another example, the delivery member shield remover 35 comprises the inclined surface 35c″ inclined towards the longitudinal axis L from the distally directed surface 35b″′ of the delivery member shield remover 35, as shown in FIG. 21C. In this example the delivery member shield remover can be assembled into the cap body 50, 51 by being inserted from the distal end of the cap body 50, 51. The inclined surface makes the delivery member shield remover 35 easily past the flexible arm 51b, then be snap-fitted to the cap body 50, 51.

Another aspect of the invention provides a medicament delivery device comprises the cap assembly as mentioned above. FIG. 1A shows a perspective view of an example of the medicament delivery device comprising an administration mechanism. An administration mechanism in this context is a mechanism or device which when the medicament delivery device is activated, shifts the medicament container and/or the medicament delivery member in the proximal direction.

The medicament delivery device 1 has a housing 3 having a proximal end 3a and a distal end 3b, and the cap assembly 5 as mentioned above. FIG. 1B shows the medicament delivery device 1 with the cap assembly 5 removed.

The medicament delivery device 1 also has an elongated medicament delivery member cover 7 which is linearly displaceable inside the housing 3, relative to the housing 3. The medicament delivery member cover 7 is biased in the proximal direction and movable from an initial extended position relative to the housing 3, in which position a proximal portion of the medicament delivery member cover 7 extends from the proximal end 3a of the housing 3, shown in FIG. 1B, to a retracted position. The direction of movement towards the retracted position is indicated by the arrow. Due to it being proximally biased, the medicament delivery member cover 7 is shifted proximally when the distal force is removed, thereby again obtaining an extended position relative to the housing 3. In one example, the medicament delivery member cover 7 is configured to operably engage with the housing 3 when reaching a final extended position after having been released from the retracted position. The medicament delivery member cover 7 is thus retained in the final extended position. In one example, the housing comprises a body 30. An opening 31 is arranged in the body 30 of the housing. Furthermore, in one example, the housing comprises a counter fixture 32 configured to be releasably engaged with the fixture 50a of the cap assembly 5.

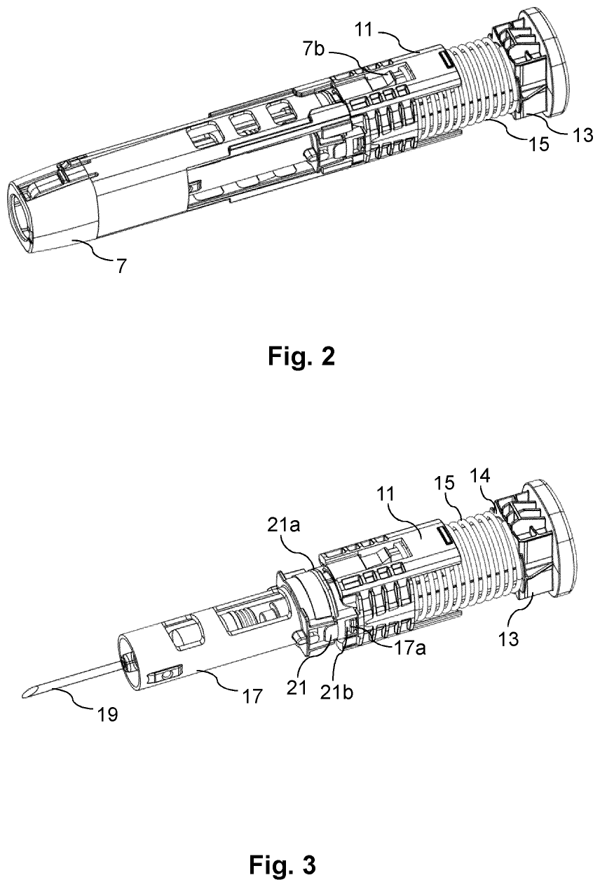

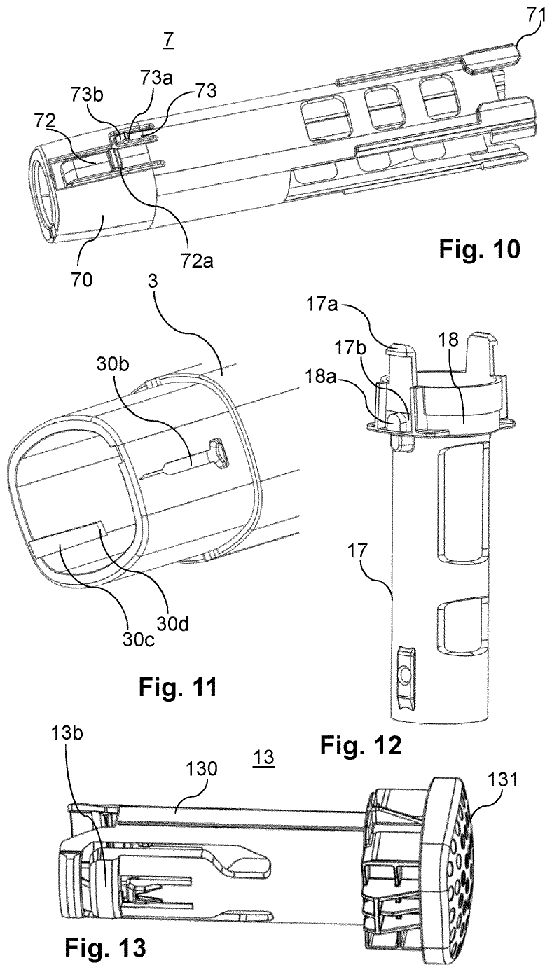

The medicament delivery device 1 also includes an administration mechanism. In FIG. 2 the housing 3 has been removed to expose an example of an administration mechanism. In one example, the administration mechanism of the medicament delivery device 1 comprises the medicament delivery member cover 7, an actuator sleeve 11, and an actuator 13 received by the actuator sleeve 11. In a preferred example, the medicament delivery member cover 7 comprises a tubular proximal portion 70 configured to surround a medicament delivery member, as shown in FIG. 10. The distal end of the medicament delivery member cover 7 is aligned with the actuator sleeve 11 and arranged proximally with respect to the actuator sleeve 11. Distal displacement of the medicament delivery member cover 7 therefore displaces the actuator sleeve 11 in the distal direction. In one example, the actuator sleeve is initially spaced apart from the medicament delivery member cover; thus, the medicament delivery member cover needs to be moved in the distal direction with a predetermined distance before encounters into the actuator sleeve. As a result, an unintentional movement of the medicament delivery member cover will not accidentally trigger the administration mechanism. Alternatively, a distal end 71 of the medicament delivery member cover 7 is initially next to the actuator sleeve 11, as shown in FIG. 15.

The actuator sleeve 11 and the medicament delivery member cover 7 are biased in the proximal direction. The administration mechanism may thus include a resilient member 15, for example a spring, arranged distally with respect to the actuator sleeve 11. The actuator 13 may according to one variation include a distal flange 14 supporting the resilient member 15. Distal displacement of the medicament delivery member cover 7 hence moves the actuator sleeve 11 distally thereby compressing the resilient member 15. The medicament delivery member cover 7 is thus urged by the resilient member 15 towards its extended position relative to the housing 3. In one example, the actuator 13 comprises a proximal portion 130 positioned within the housing 3, and a distal portion 131 position outside the distal end of the housing 3, as shown in FIG. 13. In a preferred example, the actuator 13 is snap fitted to the housing 3.

The medicament delivery member cover 7 is configured to be rotationally locked relative to the housing 3. This can for example be achieved by a structure provided on the outer surface of the medicament delivery member cover 7, configured to engage with a corresponding structure provided on the inner surface of the housing 3. Alternatively, or additionally, a cross-section of the medicament delivery member cover 7 (observing along the longitudinal axis) and a cross-section of the housing 3 (observing along the longitudinal axis) can be non-circular. For example, both medicament delivery member cover and the housing may comprise bodies respectively having rectangular cross-section.

Similarly, the actuator sleeve 11 is configured to be rotationally locked relative to the housing 3. The actuator sleeve 11 may for this purpose for example have a guide structure arranged on its outer surface and configured to engage with a corresponding guide structure provided on the inner surface of the housing 3.

The medicament delivery member cover 7 may according to one variation include a radial protrusion configured to engage with the housing 3 when the medicament delivery member cover 7 returns towards the extended position from the retracted position, to thereby become axially locked relative to the housing in a final extended position. The radial protrusion may extend radially outwards and be radially flexible, allowing it to slide into a recess or opening of the inner surface of the housing 3 thereby interlocking these two components. Alternatively, or additionally, the actuator sleeve 11 comprises the radial protrusion 7b, as shown in FIG. 2. As the medicament delivery member cover 7 is configured to be in contact with the actuator sleeve 11 when the medicament delivery member cover 7 is moved in the distal direction, once the actuator sleeve 11 is locked to the housing 3, the medicament delivery member cover 7 is also locked to the housing 3.

FIG. 3 shows the arrangement in FIG. 2 with the medicament delivery member cover 7 removed to expose a proximally displaceable medicament container holder 17 configured to accommodate a medicament container provided with a medicament delivery member, such as the needle 19 shown in the present example. In a preferred example, the medicament container holder 17 comprises a damper 18. Preferably, the damper 18 is made of resilient material, e.g., TPE. In a preferred example, the damper 18 comprises a protrusion 18a snap fits into an opening 17b of the medicament container holder 17. The damper 18 is configured to support a flange of the medicament container to avoid damage.

The administration mechanism furthermore includes a plunger holder 21 configured to be received by the actuator 13. The plunger holder 21 may be provided with a proximal flange 21a configured to bear against the medicament container arranged inside the medicament container. Proximal displacement of the plunger holder 21 hence urges the medicament container and the medicament container holder 17 in the proximal direction, providing an auto-penetration functionality.

The plunger holder 21 may also be provided with a fastener 21b configured to engage with a counter fastener 17a of the medicament container holder 17. In one example, the fastener 21b is an opening, and the counter fastener 17a is a flexible arm configured to be snap-fitted into the opening 21b. The plunger holder 21 and the medicament container holder 17 are hence axially interlocked with each other so that axial displacement in the distal direction of the plunger holder 21 also results in a corresponding distal displacement of the medicament container holder 17. In one example, the housing 3 comprises an inspection window 30e, as shown in FIG. 15, aligned with the engagement between the fastener 21b and the counter fastener 17a such that in the production line, the assembling defect can be checked via the inspection window 30e.

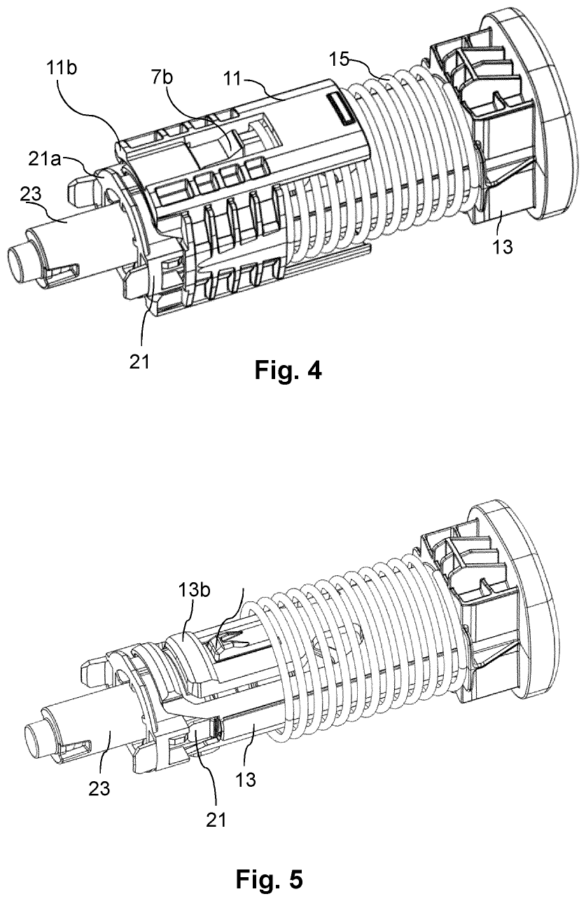

FIG. 4 shows the administration mechanism with the medicament container holder 17 removed. The exemplified administration mechanism further comprises an elongated plunger rod 23 arranged within the plunger holder 21, and a rotator not shown in FIG. 4. The actuator sleeve 11, the actuator 13, the rotator, the plunger holder 21 and the plunger rod 23 are generally concentric to one another.

FIG. 5 shows the administration mechanism without the actuator sleeve 11, exposing the actuator 13. The actuator sleeve 11 and the actuator 13 are rotationally locked relative to each other. Since the actuator sleeve 11 is configured to be rotationally locked relative to the housing 3, the actuator 13 is also rotationally locked relative to the housing 3. The actuator sleeve 11 and the actuator 13 are however configured to be axially displaceable relative to each other. The actuator 13 may thus for example comprise one or more axial ribs configured to engage with corresponding grooves of the actuator sleeve 11. Relative axial displacement may thereby be provided while preventing relative rotation between these components.

The actuator 13 has a flexible radially inwards extending arm 13b. This flexible radially inwards extending arm 13b is prevented from flexing radially outwards by the inner surface of the actuator sleeve 11 when the actuator sleeve 11 is in its initial position. In particular, the inner surface of the actuator sleeve 11 bears against the flexible radially inwards extending arm 13b such that the arm 13b bears against a radial surface of the plunger holder 21 in the initial extended position of the medicament delivery member cover 7, as will be explained in more detail in the following.

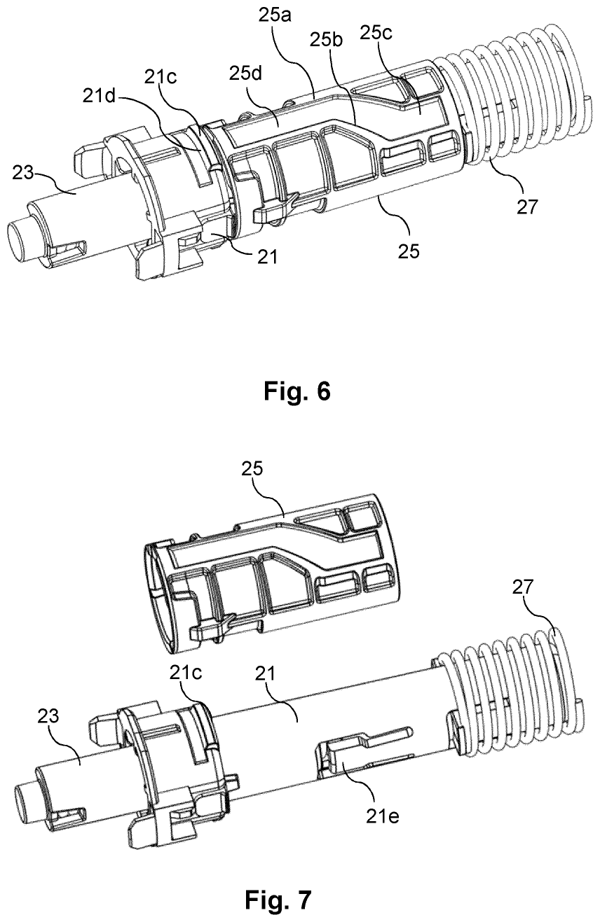

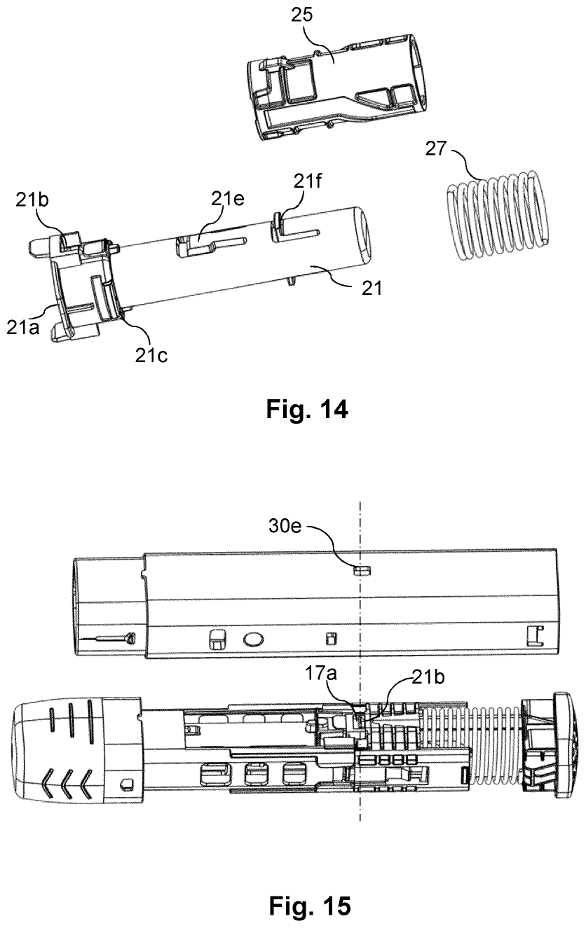

In FIG. 6, the actuator 13 has been removed to expose the rotator 25 which is arranged around the plunger holder 21 and to show a proximal portion of the plunger holder 21. The plunger holder 21 has a radially extending flange 21c having a proximally directed surface 21d. The flexible radially inwards extending arm 13b bears against this proximally directed surface 21d in the initial extended position of the medicament delivery member cover 7. Moreover, the plunger holder 21 is proximally biased by another resilient member 27, arranged inside the actuator. The engagement between the flexible radially inwards extending arm 13b and the proximally directed surface 21d prevents the plunger holder 21 from proximal displacement due to the biasing as long as the actuator sleeve 11 prevents the flexible radially inwards extending portion 13b from flexing radially outwards. In a preferred example, the plunger holder 21 comprises a distally directed surface adjacent to the resilient member 27. In a preferred example, the plunger holder 21 comprises a flexible arm 21f having the distally directed surface, as shown in FIG. 14. In this example, the rotator 25 can be assembled to the plunger holder 21 by passing through the distal end of the plunger holder 21. The flexible arm 21f is deformed inwardly by the rotator 25 when the rotator passes by, then flexes back to its initial configuration. Thus, the rotator 25 is positioned between the flange 21c and the flexible arm 21f, as shown in FIG. 6.

The rotator 25 bears against a distally directed surface of the plunger holder 21, which prevents the rotator 25 from independently moving proximally relative to the plunger holder 21. In one example, the flange 21c comprises the distally directed surface of the plunger holder 21. The rotator 25 is also proximally biased. In particular, the rotator 25 is proximally biased by the resilient member 27.

The rotator 25 has a guide structure 25a. In one example, the guide structure 25a is formed by a recess/cut-out in a wall of the rotator 25 facing in a direction away from the longitudinal axis L, e.g., facing outwards. The guide structure 25a includes a proximal portion 25d defined by an axial groove portion and a contiguous proximal portion 25b defined by an inclined groove portion. The inclined groove portion is inclined with respect to a plane extending through the centre of the rotator 25 and parallel with the axial groove portion. The guide structure 25a are dimensioned to receive a protrusion 13c of the actuator 13. In one example, the protrusion of the actuator 13 is the flexible radially inwards extending arm. Alternatively, the protrusion 13c of the actuator 13 is extending from a second flexible arm, as shown in FIG. 9a. As a result, when the plunger holder 21 is released and thus, axially moves relative to the actuator 13, the rotator 25 is moved in the distal direction by the resilient member 27 together with the plunger holder 21, the protrusion 13c will reach the inclined groove portion, forcing the rotator 25 to rotate.

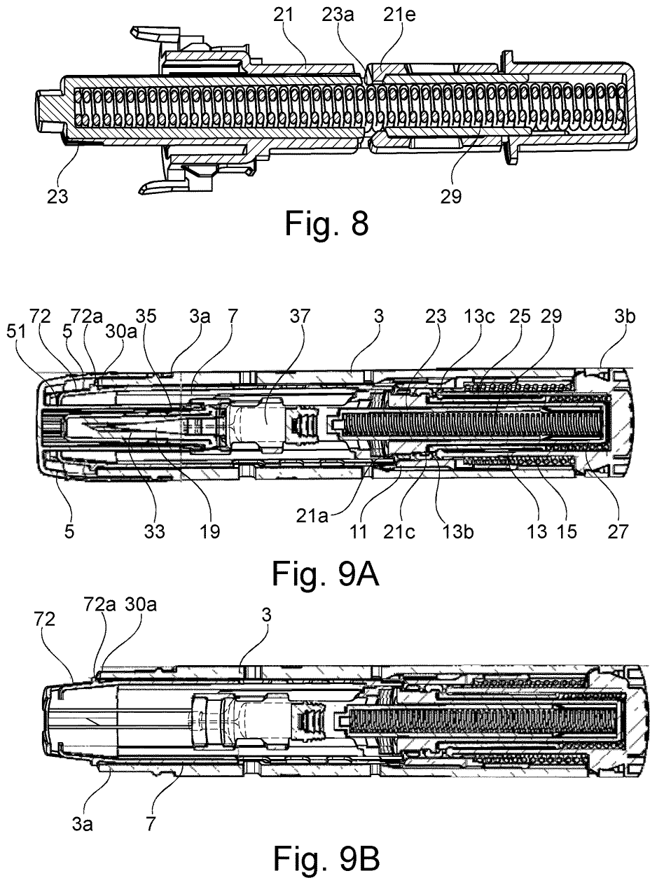

In FIG. 7, the rotator 25 has been moved away from the plunger holder 21 to expose the plunger holder 21. The plunger rod 23 is configured to be proximally biased by yet another resilient member 29 as shown in FIG. 8. In one example, this resilient member 29 is arranged inside the hollow plunger rod 23. Alternatively, the resilient member can be positioned between a distal end of the plunger rod and a proximally directed surface of the plunger holder; thus, the plunger rod doesn't need to be hollow. The plunger holder 21 is configured to initially engage with the plunger rod 23 to prevent the plunger rod 23 from axial displacement in the proximal direction. Hereto, according to one variation the plunger holder 21 has one or more flexible radially inwards extending tab(s) 21e configured to engage with a corresponding radial opening(s) 23a of the plunger rod 23, as shown in FIG. 8. In one example, the rotator 25 comprises a support wall extending from an inner surface of the rotator 25 towards the longitudinal axis L. The support wall is configured to prevent the flexible radially inwards extending tab 21e from flexing radially outwards so that it engages with the radial opening 23a of the plunger rod 23. When the rotator 25 is rotated, the support wall is no longer radially aligned with the flexible radially inwards extending tab(s) 21. The plunger rod 23 is thereby released from its engagement with the plunger holder 21 allowing it to move proximally so that medicament expulsion may be commenced. Thus, the plunger rod 23 can be biased towards the proximal end of the housing 3 by the resilient member 29. Alternatively, the rotator may be provided with a window, i.e. an opening configured to align with the flexible radially inwards extending tab 21e when the rotator is rotated.

FIG. 9A shows a longitudinal section of the medicament delivery device 1 in an initial state in which the cap assembly 5 is positioned proximally relative to the housing 3 to protect the internal components of the medicament delivery device 1. In this position, a number of components are biased in the proximal direction. In particular, the resilient member 27, termed a first resilient member, is configured to bias the plunger holder 21 and the rotator 25 proximally, resilient member 29, termed a second resilient member, is configured to bias the plunger rod 23 proximally, and resilient member 15, termed a third resilient member, is configured to bias the medicament delivery member cover 7 proximally. According to the present example, each resilient member 15, 27, 29 is a spring.

The exemplified medicament delivery device 1 accommodates a medicament container 37, a medicament delivery member in the form of a needle 19, and a delivery member shield 33, i.e. a needle shield. The cap assembly 5 is provided with the delivery member shield remover 35 for removing the delivery member shield 33 when the cap assembly 5 is removed, as mentioned above. It should be noted that the medicament delivery member might be a spray nozzle, e.g., when the medicament delivery device is a jet injector, that needs to be sealed before use. Thus, the delivery member shield and therefore the delivery member shield remover might be needed for those medicament delivery devices without needle.

Turning now to FIG. 9B, the medicament delivery device 1 is again in the initial position, however with the cap assembly 5 removed. The delivery member shield 33 has thus been removed by the delivery member shield remover 35. The medicament delivery member cover 7 extends from the proximal end 3a of the housing 3 and is arranged in the extended position. Moreover, the actuator sleeve 11 is arranged in its initial position. In this state, the medicament delivery device 1 is ready for injection, activation being initiated by fully pushing the medicament delivery member cover 7 distally into the housing 3, the direction being indicated by the arrow.

Once the medicament delivery member cover 7 has been pushed distally into the housing 3, the medicament delivery member cover 7 has hence been moved linearly inside the housing 3. The distal end 71 of the medicament delivery member cover 7 is thereby brought towards and into contact with the proximal end of the 11b of the actuator sleeve 11, thereby displacing the actuator sleeve 11 in the distal direction.

In a preferred example, the actuator sleeve 11 has an inner dimension, namely the distance between two opposite inner surfaces which prevent the flexible radially inwards extending arm 13b of the actuator 13 from flexing radially outwards, which is smaller than the corresponding inner dimension of the medicament delivery member cover 7. Hereto, when the actuator sleeve 11 has been displaced sufficiently far distally by the medicament delivery member cover 7, the medicament delivery member cover 7 will be axially aligned with, the flexible radially inwards extending arm 13b. Because of the larger inner dimension of the medicament delivery member cover 7, the flexible radially inwards extending arm 13b is allowed to flex radially outwards as it is being urged by the proximally biased plunger holder 21. In this manner, the radial flange 21c is allowed to pass underneath the flexible radially inwards extending arm 13b. The plunger holder 21 is thus released from being axially interlocked with the actuator 13 and moved proximally due to the proximal force exerted by the first resilient member 27.

When the rotator 25 moves together with the plunger holder 21 in the proximal direction relative to the actuator 13, the protrusion 13c is hence moved into the guide structure 25a. The protrusion 13c is hence moved in the guide structure 25a, from the proximal portion into a distal portion 25c. As previously mentioned, the plunger holder 21 is configured to displace the medicament container 37 proximally since the proximal flange 21a of the plunger holder 21 bears against a distal flange 37a of the medicament container 37.

The axial length of the proximal portion 25b of the guide structure 25a determines the amount that the plunger holder 21 is moved proximally and thus the amount that the medicament container 37 is moved proximally before medicament expulsion can commence. The axial length may in particular be dependent on a particular application of the medicament delivery device 1. According to the present example, the medicament container 37 is moved proximally to such an extent that the needle 19 is shifted proximally to extend fully beyond the proximal end 3a of the housing 3.

When the protrusion 13c moves into the distal portion 25c, the rotator 25 is rotated, since the actuator 13 is rotationally locked relative to the housing 3. When the rotator is rotated, the support surface of the rotator is no longer aligned with the flexible radially inwards extending tab 21e. The flexible radially inwards extending tab 21e will thereby be allowed to flex radially as a result of the proximally directed force provided by the resilient member 29.

The plunger rod 23 is thereby released from its engagement with the plunger holder 21, allowing it to move proximally as a result from it being biased proximally by the resilient member 29. Upon being released from engagement with the plunger holder 21, the plunger rod 23 is configured to proximally displace a plunger 38 arranged in the medicament container 37, and to move it proximally until the entire dose of medicament has been expelled through the needle 19.

Once the medicament delivery operation is completed, the user of the medicament delivery device is instructed to lift the medicament delivery member cover 7 from a medicament delivery site. Thus, the medicament delivery member cover 7 is moved proximally until the radial protrusion 7b engages with the opening 3c of the housing 3. The medicament delivery member cover 7 will thereby interlock with the housing 3 (via the engagement between the protrusion 7b of the actuator sleeve 11) and is thus prevented from being subsequently being moved distally towards the retracted position. The used needle 19 will thus be covered by the medicament delivery member cover 7 ensuring anyone handling the used medicament delivery device 1 from being pierced by it.

Furthermore, according to another aspect of the invention, a safety mechanism of the medicament delivery device can be provided. The safety mechanism is configured to prevent the administration mechanism being accidentally trigger before the user plans to use the medicament delivery device, e.g., when the medicament delivery device falls.

As mentioned above, the initial distal displacement of the medicament delivery member cover 7 is configured to trigger the administration mechanism of the medicament delivery device, thus, the safety mechanism is provided by limiting the distal displacement of the medicament delivery member cover 7. In this example, the medicament delivery member cover comprises a flexible arm 72 comprising a radially outwards extending protrusion 72a, as shown in FIGS. 1B, 9A-9B, and 10. When cap assembly 5 is attached to the housing 3, as shown in FIG. 9A, the flexible arm 72 of the medicament delivery member cover 7 is blocked from flexing radially inwards by the inner body of the cap assembly 5. As a result, the radially outwards extending protrusion 72a is blocked from moving towards the distal end of the housing 3 by a proximally directed surface 30a of the housing 3. Thus, before the cap assembly 5 is removed from the housing 3, the medicament delivery member cover 7 is prevented from moving in the distal direction relative to the housing 3 such that the accidental trigger of the administration mechanism can be prevented. Once the user plans to use the medicament delivery device, the user removes the cap assembly 5. As the flexible arm 72 of the medicament delivery member cover 7 is no longer blocked from flex radially inwards, the distal displacement of the medicament delivery member cover 7 deforms the flexible arm 72 when the radially outwards extending protrusion 72a is in contact with the proximally directed surface 30a of the housing 3.

Furthermore, in a preferred example, the medicament delivery member cover 7 comprises a distal block 73a, preferably, is a protrusion extending from the proximal portion 70 of the medicament delivery member cover, as shown in FIG. 10. The distal block 73a is configured to be positioned in a recess 30c in a wall of the housing 3. A distal end 30d of the recess 30c is configured to define a maximum movable distance of the medicament delivery member cover 7 in the distal direction relative to the housing 3. In a preferred example, the distal block 73a is extending from a second flexible arm 73. Furthermore, a proximal end 73b of the distal block 73a is a chamfer surface facing towards the proximal end of the housing 3. As result, the medicament delivery member cover 7 can be assembled, in the production line, into the housing 3 by being inserted from the distal end of the housing 3 until the distal block 73a is positioned within the recess 30c.

Furthermore, the housing of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

Other aspects of the invention are defined by the following clauses.
1. A cap assembly of a medicament delivery device, the cap assembly comprising:
   a cap body extending along a longitudinal axis between a proximal end and a distal end;
   a delivery member shield remover extending along the longitudinal axis between a proximal end and a distal end;
   wherein the delivery member shield remover is positioned within the cap body; wherein the delivery member shield remover comprises a grabber configured to be engaged with a delivery member shield of the medicament delivery device such that a user of the medicament delivery device can remove the delivery member shield when remove the cap assembly from a housing of the medicament delivery device;
   wherein the cap body comprises a proximally directed surface adjacent to a distally directed surface of the delivery member shield remover;
   wherein the cap body comprises a user-accessible surface configured to be gripped by the user of the medicament delivery device such that the user can remove the cap assembly from the housing of the medicament delivery device via gripping on the user-accessible surface; wherein the proximally directed surface and the user-accessible surface are immovable relative to one another; and
   wherein the distally directed surface of the delivery member shield remover is positioned closer to the distal end of the delivery member shield remover than the proximal end of the delivery member shield remover.
2. The cap assembly according to clause 1, wherein the distal end of the delivery member shield remover comprises the distally directed surface of the delivery member shield remover.
3. The cap assembly according to clause 1 or 2, wherein the grabber of the delivery member shield remover comprises a proximally directed surface configured to be engaged with the delivery member shield of the medicament delivery device.
4. The cap assembly according to clause any one of the preceding clauses, wherein the delivery member shield remover comprises at least two grabbers lined up to one another in the direction of the longitudinal axis.
5. The cap assembly according to any one of the preceding clauses, wherein the cap body comprises a distally directed surface adjacent to the proximal end of the delivery member shield remover.
6. The cap assembly according to any one of the preceding clauses, wherein the cap body comprises a flexible arm extending towards the proximal end of the cap body; and wherein the flexible arm comprises the proximally directed surface of the cap body such that the delivery member shield remover is attached to the cap body via a snap fit.
7. The cap assembly according to any one of the preceding clauses, wherein the cap body comprises an inner body and an outer body being coaxial to the inner body; wherein the outer body comprises the user-accessible surface; and wherein the inner body comprises the distally directed surface.
8. The cap assembly according to clause 7, wherein the outer body comprises a proximal wall extending in a direction transverse to the longitudinal axis; wherein the inner body comprises a body wall extending from the proximal wall of the outer body along the longitudinal axis towards the distal end of the cap body; wherein the body wall of the inner body defines a cannel; and wherein the delivery member shield remover is snuggly fit into the cannel of the inner body.
9. The cap assembly according to clause 8, wherein the inner body comprises a rib extending from the body wall towards the longitudinal axis; and wherein the rib comprises the distally directed surface of the cap body.
10. The cap assembly according to clause 8 when dependent on clause 6 or clause 9 when dependent on clause 6, wherein the inner body comprises the flexible arm protruding from the body wall.
11. The cap assembly according to clause 10, wherein a cut-out/recess is arranged in the body wall of the inner body; and wherein the flexible arm extends from an edge of the cut-out/recess towards the proximal end of the cap body.
12. The cap assembly according to a combination of clause 9, wherein the delivery member shield remover is positioned between the rib of the inner body and the flexible arm of the inner body.
13. The cap assembly according to any one of clauses 7 to 12, wherein the outer body comprises a fixture configured to be releasably attached to the housing of the medicament delivery device.
14. The cap assembly according to any one of the preceding clauses, wherein the grabber is positioned closer to the distal end of the delivery member shield remover than the proximal end of the delivery shield remover.
15. The cap assembly according any one of the preceding clauses, wherein the delivery member shield remover comprises an inclined surface inclined towards the longitudinal axis from the distally directed surface of the delivery member shield remover.
16. The cap assembly according to any one of the preceding clauses, wherein the delivery member shield remover comprises a ledge extending radially outward that comprises the distally directed surface.
17. The cap assembly according to clause 16, wherein the ledge is a rim circumferentially extends around the delivery member shield remover.

1-15. (canceled)

16. A cap assembly of a medicament delivery device, the cap assembly comprising:
a cap body extending along a longitudinal axis between a proximal end and a distal end; and
a delivery member shield remover extending along the longitudinal axis between a proximal end and a distal end,
wherein the delivery member shield remover is positioned within the cap body,
wherein the delivery member shield remover comprises a grabber configured to be engaged with a delivery member shield of the medicament delivery device such that a user of the medicament delivery device can remove the delivery member shield when remove the cap assembly from a housing of the medicament delivery device,
wherein the cap body comprises a proximally directed surface adjacent to a distally directed surface of the delivery member shield remover,
wherein the cap body comprises a user-accessible surface configured to be gripped by the user of the medicament delivery device such that the user can remove the cap assembly from the housing of the medicament delivery device via gripping on the user-accessible surface,
wherein the proximally directed surface and the user-accessible surface are immovable relative to one another, and
wherein the distally directed surface of the delivery member shield remover is positioned closer to the distal end of the delivery member shield remover than the proximal end of the delivery member shield remover.

17. The cap assembly according to claim 16, wherein the distal end of the delivery member shield remover comprises the distally directed surface of the delivery member shield remover.

18. The cap assembly according to claim 16, wherein the grabber of the delivery member shield remover comprises a proximally directed surface configured to be engaged with the delivery member shield of the medicament delivery device.

19. The cap assembly according to claim 16, wherein the delivery member shield remover comprises at least two grabbers lined up to one another in the direction of the longitudinal axis.

20. The cap assembly according to claim 16, wherein the cap body comprises a distally directed surface adjacent to the proximal end of the delivery member shield remover.

21. The cap assembly according to claim 16, wherein the cap body comprises a flexible arm extending towards the proximal end of the cap body, and wherein the flexible arm comprises the proximally directed surface of the cap body such that the delivery member shield remover is attached to the cap body via a snap fit.

22. The cap assembly according to claim 16, wherein the cap body comprises an inner body and an outer body being coaxial to the inner body, wherein the outer body comprises the user-accessible surface, and wherein the inner body comprises the distally directed surface.

23. The cap assembly according to claim 22, wherein the outer body comprises a proximal wall extending in a direction transverse to the longitudinal axis, wherein the inner body comprises a body wall extending from the proximal wall of the outer body along the longitudinal axis towards the distal end of the cap body, wherein the body wall of the inner body defines a channel, and wherein the delivery member shield remover is snuggly fit into the channel of the inner body.

24. The cap assembly according to claim 23, wherein the inner body comprises a rib extending from the body wall towards the longitudinal axis, and wherein the rib comprises the distally directed surface of the cap body.

25. The cap assembly according to claim 24, wherein the delivery member shield remover is positioned between the rib of the inner body and the flexible arm of the inner body.

26. The cap assembly according to claim 22, wherein the outer body comprises a fixture configured to be releasably attached to the housing of the medicament delivery device.

27. The cap assembly according to claim 16, wherein the grabber is positioned closer to the distal end of the delivery member shield remover than the proximal end of the delivery shield remover.

28. The cap assembly according to claim 16, wherein the delivery member shield remover comprises an inclined surface inclined towards the longitudinal axis from the distally directed surface of the delivery member shield remover.

29. The cap assembly according to claim 16, wherein the delivery member shield remover comprises a ledge extending radially outward that comprises the distally directed surface.

30. The cap assembly according to claim 29, wherein the ledge is a rim circumferentially extends around the delivery member shield remover.
